(19) United States
(12) Patent Application Publication
(10) Pub. No.: US 2024/0261470 A1
(43) Pub. Date: Aug. 8, 2024

(54) COMPRESSION-BASED MEDICANT RELEASING IMPLANTS FOR MENICSCAL REPLACEMENT AND METHODS FOR IMPLANTATION AND REFILLING THEREOF

(71) Applicant: Medos International Sàrl, Le Locle (CH)

(72) Inventors: Ravi PATEL, East Providence, RI (US); Jordan JACOBS, Randolph, MA (US); Michael J. ROMANKO, Raynham, MA (US); Austin BRIDGES, Boston, MA (US); Terry DAGLOW, Raynham, MA (US); Frederick E. SHELTON, IV, Hillsboro, OH (US); Jason L. HARRIS, Lebanon, OH (US)

(21) Appl. No.: 18/106,115

(22) Filed: Feb. 6, 2023

(51) Int. Cl. A61L 27/36; A61L 27/54

(52) U.S. Cl. CPC A61L 27/3654; A61L 27/54; A61L 2300/602

(57) ABSTRACT

An implant for releasing one or more substances within the body is provided. The implant can include a scaffold configured to be implanted within the body to provide a foundation for an implant that provides structural support for promoting meniscus repair and regeneration. The scaffold can be loaded with medicants and/or analgesics that are capable of being released from the implant following implantation to promote healing and reduce inflammation post-surgery. The scaffold can be refilled with substances once implanted. For example, the implant can be connected to an access port that is embedded in the skin to allow refilling of the scaffold. In some embodiments, the scaffold can include a plurality of chambers that are configured to retain a separate substances therein.

### FIELD

The application relates generally to devices and methods for anchoring an implant containing a compression-based medicant into a joint space, and more particularly, for securing, filling, and/or refilling implants for meniscal replacement with one or more compounds to promote healing thereof.

### BACKGROUND

A common injury, especially among athletes and people of advanced age, is the complete or partial tears of tendons, ligaments, or other soft tissues in the knee. An example of these soft tissues are the two menisci located in each knee: the medial meniscus and the lateral meniscus. They are paired crescent fibrocartilaginous wedges positioned between the femoral condyle and tibial plateau. They are firmly anchored onto the tibial plateau and confined within the tibiofemoral compartment of the joint capsule. Each meniscus plays a critical role in maintaining the homeostasis, biomechanics, and structural stability of the knee joint. Unfortunately, the menisci are predisposed to damage. Specifically, tears of one or both menisci of the knee can significantly affect the ability of the knee to perform its typical functions. Further, injuries to the menisci can take a long time to heal, and, in at least some instances, can fail to heal on their own. For example, self-healing of injured adult menisci can only occur in the peripheral vascularized portion, while the spontaneous repair of the inner avascular region occurs infrequently, if ever.

Current solutions that aim to repair, replace, and regenerate menisci exhibit several shortcomings. First, anatomically, each meniscus is typically perfectly congruent, or at least substantially congruent, with the femoral condyle and the tibial plateau. Such congruency helps enable critical functions such as loadbearing, shock absorption, and lubrication of the meniscus in the knee joint. Following injury, this congruence is often impacted, with surgical repairs and replacements failing to perfectly, or even near perfectly, replicate the alignment of the meniscus in between the femur and the tibia. This failure results in improper cushioning between the femur and tibia, and eventual wear and degradation of one or both bones.

Second, meniscal repair procedures are difficult and time-intensive, with the quality of conventional implants deteriorating over time. In existing procedures, lubricant associated with the implants, introduced in conjunction with surgical repairs and replacements, can deteriorate. Additionally, or alternatively, site pain at the location the implant can often occur due, for instance, to misalignment between the meniscus and the bones. Because of these deteriorations and other complications, additional surgical procedures are often needed to refill the implant and/or the surrounding spaces with lubricants and/or other medicines. Additional procedures result in additional trauma to the tissue, and often lead to extended recovery times due, at least in part, to repeated intrusion into surrounding tissues.

Still further, existing implants are limited in their ability to perform either as old menisci performed, and/or in a manner that would be beneficial to the patient. Existing implants can fail to provide a desired strength suitable for continued use by the patient. Existing implants also lack the ability to provide for easy adjustability, either during the implantation procedure or after such surgery. The ability to make adjustments in one or both scenarios may be beneficial, especially post-surgery for younger patients that may still experience growth of the skeletal system.

Accordingly, there is a need for improved devices and methods for implants for meniscal replacement that allow for customization and adjustments following implantation.

### SUMMARY

Examples of the present disclosure include devices and methods for performing surgical procedures involving the knee and its surrounding joint space, such as meniscal anchoring, replacement, and re-implantation, among other repair procedures. More specifically, certain examples of the devices and methods disclosed herein allow a user to introduce an implant having medicant-releasing capabilities to into a body of a patient. Representative examples of the implant can include a scaffold configured to be anchored into the body for releasing one or more substances contained in the scaffold within the joint space. The scaffold can be preloaded with the substances and/or loaded in situ such that the substances can be released from the scaffold in response to a compressive force. In some embodiments, the scaffold can be configured to be introduced in an uninflated state and inflated in situ to form the implant.

The scaffold can be refilled with substances once implanted. For example, the implant can be connected to an access port that can be embedded in the skin to allow refilling of the scaffold. A pump can be in fluid communication with the scaffold to adjust flow rate of the substances to the implant. In some embodiments, the scaffold can include a plurality of chambers, with each chamber configured to retain a specific substance therein. The chambers can be separated such that the substances contained therein do no mix and each chamber can elute the specific substance when compressed. In addition, the body of the scaffold can include a channel for distributing the one or more substances therethrough to facilitate filling of each chamber. One or more pathways can branch from the channel to deliver the substances to each chamber. Alternatively, or additionally, the implant can be manufactured by electrospinning in which charged fibers of polymer melts are drawn to form an implant resembling a meniscus.

One exemplary surgical method includes accessing a joint space of a patient that comprises, or previously comprised, a meniscus. The method also includes delivering a scaffold to one of replace the meniscus or support the meniscus, with the scaffold including an internal cavity and the scaffold having one or more substances disposed in a permeable portion of the internal cavity at least one of during or after it is delivered to the joint space, and anchoring the scaffold to the tibia. The scaffold is configured to release the one or more substances from the internal cavity of the scaffold, to the joint space, in response to one or more compressive forces exerted onto the scaffold.

In some embodiments of the method, the internal cavity can include the permeable portion and a non-permeable portion. The method can further include loading the permeable portion of the internal cavity of the scaffold with the one or more substances. Loading the permeable portion of the internal cavity of the scaffold with the one or more substances can occur in situ. In some embodiments, the method can further include delivering the one or more substances to the internal cavity after the scaffold is anchored to the tibia.

The method can further include delivering the scaffold into the joint space in an uninflated state. Inflating the scaffold can form the implant that accommodates the joint space. In some embodiments, the method can further include coupling an access port to a location on the patient that is remote from the joint space, with the access port being in fluid communication with the scaffold to deliver the one or more substances to the scaffold. Delivering the scaffold can include electrospinning the scaffold. Electrospinning the scaffold can further include operating an electrospinning device to deliver material that forms the scaffold to the joint space.

In some embodiments, the method can further include controlling at least one of a temperature of a material used to form the scaffold, with a diameter of a nozzle that delivers a material used to form the scaffold, or a flow rate of a material used to form the scaffold. The scaffold can be configured to release the one or more substances substantially continuously. In some embodiments, the scaffold can be configured to release the one or more substances in a time-released manner.

The method can further include that the scaffold can be configured to release the one or more substances based on movement of a patient in which the scaffold is located and/or a continuous feedback loop associated with movement of the patient in which the scaffold is located. In some embodiments, the scaffold can be configured to release an amount of the one or more substances that is proportional to the one or more compressive forces exerted on the scaffold. In some embodiments, the scaffold can be configured to adjust the release of the one or more substances in response to different compression pressure gradients experienced by the scaffold. The scaffold can be configured to be located in the joint space for at least one year.

One exemplary embodiment of a meniscal surgical implant includes a scaffold. The scaffold includes a body portion disposed between a first end of the body portion and a second end of the body portion. The body portion is sized and shaped for use as one of a replacement or supplement of a meniscus in a patient, with the body portion defining an internal cavity that is configured to have one or more substances disposed in the internal cavity. The body portion has a porous outer surface configured to allow secretion of one or more substances disposed in the internal cavity to an environment outside of the body portion, with the environment being proximate to one of a location where the meniscus was previously or the meniscus in the patient. The scaffold is configured to release one or more substances from the internal cavity of the body portion in response to one or more compressive forces applied to the body portion by way of anatomy proximate to the one of the location where the meniscus was previously or the meniscus of the patient.

The implant can further include that the scaffold is C-shaped to mimic the shape of the meniscus. The scaffold can be configured to conform to a space in which it is disposed. In some embodiments, the scaffold can be configured to be anchored to the tibia. The internal cavity and/or deformable external cavity can be expandable and the body portion further includes one or more filling openings for receiving at least one of a gas or a liquid to inflate the internal cavity. The implant can further include one or more substances disposed within the scaffold, with the one or more substances comprising one or more of a medicant or hyaluronic acid (HA).

The medicant can include at least one of: an analgesic, a non-steroidal inflammatory drug (NSAID), or a non-opioid. In some embodiments, the scaffold can be configured to release the one or more substances substantially continuously. The scaffold can be configured to release the one or more substances in a time-released manner. In some embodiments, the scaffold can be configured to release the one or more substances based on movement of a patient in which the scaffold is located and/or a continuous feedback loop associated with movement of the patient in which the scaffold is located. The scaffold can be configured to release an amount of the one or more substances that is proportional to the one or more compressive forces exerted on the scaffold. The scaffold can be configured to adjust the release of the one or more substances in response to different compression pressure gradients experienced by the body portion.

In some embodiments, the scaffold further includes a plurality of chambers formed in the scaffold, with each of the plurality of chambers being configured to receive at least one substance of the one or more substances. One or more non-porous walls can be disposed between chambers of the plurality of chambers such that the at least one substance received in a first chamber of the plurality of chambers is not able to pass through the one or more non-porous walls to a second chamber of the plurality of chambers. The scaffold can further include a channel that extends from a location proximate to the first end of the body portion towards a location that is more proximate to the second end of the body portion than the first end is, with the channel being in fluid communication with one or more branched pathways configured to deliver the one or more substances to the plurality of chambers formed in the body portion. In some embodiments, one or more dispersing openings can be configured to allow the one or more substances to be released from the internal cavity in response to the one or more compressive forces applied to the body portion by way of anatomy proximate to the one of the location where the meniscus was previously or the meniscus of the patient.

The implant can further include an access port and a tube coupled to the access port and can provide fluid communication between the access port and the body portion to allow the one or more substances to be introduced into the internal cavity. The access port can be configured to be implanted at least one of on or proximate to a skin surface of a patient. The scaffold can be configured to be in fluid communication with a pump to regulate a flow of the one or more substances to the scaffold. In some embodiments, the scaffold is configured to be located at the location of the replacement or supplement of the meniscus in the patient for at least one year.

### BRIEF DESCRIPTION OF DRAWINGS

This disclosure will be more fully understood from the following detailed description taken in conjunction with the accompanying drawings, in which:

FIG. 1A is a schematic top cross-sectional view of a prior art tibia having the medial and lateral menisci positioned on the medial and lateral plateaus, respectively;

FIG. 1B is a schematic top cross-sectional view of a prior art tibia having the medial and lateral menisci removed from the medial and lateral plateaus;

FIG. 2 is a transparent front view of a knee having a meniscal implant of the present embodiments positioned therein;

FIG. 3A is a perspective view of one exemplary embodiment of a scaffold of the implant of the present embodiments showing forces acting thereon;

FIG. 3B is a side view of the scaffold of FIG. 3A, schematically illustrated as being positioned in a joint space;

FIG. 3C is a top perspective view of another exemplary embodiment of a scaffold of the implant of the present embodiments, the scaffold having a plurality of openings formed therein;

FIG. 3D is a perspective view of the scaffold of FIG. 3C;

FIG. 3E is a bottom perspective view of the scaffold of FIG. 3C;

FIG. 4A is a perspective view of another exemplary embodiment of a scaffold in an uninflated state and positioned on the tibia;

FIG. 4B is the perspective view of the scaffold of FIG. 4A, the scaffold being in an inflated state to form a meniscal implant;

FIG. 5 is the side view of the scaffold of FIG. 3B having one or more substances disposed therein for release;

FIG. 6A is a schematic side cross-sectional view of a system that includes the scaffold of FIG. 3A implanted in the body, the scaffold being in fluid communication with an access port associated with a leg of a patient;

FIG. 6B is a schematic front view of the access port of FIG. 6A;

FIG. 6C is a schematic side view of a syringe configured to be received within the access port of FIG. 6B to deliver a medicant to the scaffold of FIG. 6A;

FIG. 7A is a schematic side view of a pump associated with a system that includes the scaffold of FIG. 3A implanted in the body, the scaffold being in fluid communication with an access port associated within a leg of a patient;

FIG. 7B is a perspective view of the access port of FIG. 7A, the access port having a septum formed thereon;

FIG. 8A is a perspective view of another exemplary embodiment of a system that includes a scaffold having a plurality of chambers, the scaffold being in communication with an access port;

FIG. 8B is a cross-sectional view of the access port of FIG. 8A taken along line B-B;

FIG. 8C is a cross-sectional view of the system of FIG. 8A taken along line C-C;

FIG. 8D is a magnified view of the cross-sectional view of the system of FIG. 8C;

FIG. 9A is a perspective view of an electrospinning device inserted through a cannula;

FIG. 9B is a perspective view of the electrospinning device of FIG. 9A actuated to draw charged fibers of polymer melts;

FIG. 9C is a perspective view of the electrospinning device of FIG. 9A continuing to draw the charged fibers of polymer melts while being retracted through the cannula;

FIG. 9D is a perspective view of the electrospinning device of FIG. 9A continuing to draw the charged fibers of polymer melts while being retracted through the cannula;

FIG. 9E is a perspective view of the electrospinning device of FIG. 9A continuing to draw the charged fibers of polymer melts while being retracted through the cannula;

FIG. 9F is a perspective view of the electrospinning device of FIG. 9A continuing to draw the charged fibers of polymer melts while being retracted through the cannula;

FIG. 9G is a perspective view of the electrospinning device of FIG. 9A forming a scaffold using the charged fibers of polymer melts while being retracted through the cannula; and

FIG. 10 is a flow diagram illustrating a method for electrospinning a meniscus, for example using the device of FIG. 9A.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and/or the methods and procedures in which the systems and devices will be used. To the extent the term “proximate” is used to describe a location, a person skilled in the art will appreciate what a suitable nearby location is encompassed by use of the term with respect to the particular location being described. Unless indicated otherwise, use of the term “proximate” is inclusive of “at,” meaning if a location is described as being “proximate” to a joint space, it can be nearby and/or at the joint space.

The figures provided herein are not necessarily to scale. Further, to the extent arrows are used to describe a direction a component can expand or move, these arrows are illustrative and in no way limit the direction the respective component can expand or move. A person skilled in the art will recognize other ways and directions for creating the desired tension or movement. Likewise, while in some embodiments movement of one component is described with respect to another, a person skilled in the art will recognize that other movements are possible. Additionally, a number of terms may be used throughout the disclosure interchangeably but will be understood by a person skilled in the art. By way of non-limiting example, the terms “substances” and “compounds” may be used interchangeably with one another to refer to solid, liquid, and/or gaseous materials that are passed through an access port to the scaffold of the present embodiments. Moreover, mention of the term “compounds” may be a reference to a single substance or material or a plurality of substances or materials combined together. Additionally, a person skilled in the art will recognize that the terms “scaffold” and “implant” can refer to the same entity, with “scaffold” referring to an unfilled body prior to being delivered into the body, and “implant” referring to the scaffold once anchored into the body and filled with one or more substances to accommodate the space into which it is inserted.

The present disclosure is generally directed to devices and methods for introducing surgical implants into the body, and more particularly, devices and methods for meniscal replacement and anchoring of the same. Surgical implants described herein generally include a scaffold configured to be implanted within the body to provide a foundation for an implant that provides structural support for promoting meniscus repair and regeneration. The scaffold can be impregnated, injected, and/or inflated with one or more substances in situ to form the implants within the joint space. The substances can include medicants and/or analgesics that are capable of being released from the implant following implantation to promote healing and reduce inflammation post-surgery. In some embodiments, the scaffold can be coupled to an access port that can be implanted within or on the skin of a patient (sometimes referred to as proximate to the skin surface), or otherwise associated with the body of the patient, to deliver substances to the implant without further surgical intervention. Examples of the present disclosure also include methods of fabricating the scaffolds are also disclosed, with these methods including electrospinning nanofibrous materials to recreate the order of fibrous structures of typical dense connective tissues such as tendons and menisci to approximate the microenvironment of the native extracellular matrix, in which meniscal cells reciprocally interact with surrounding nanofibers, mimicking physiological interactions between the extracellular matrix of the meniscus and its resident cells.

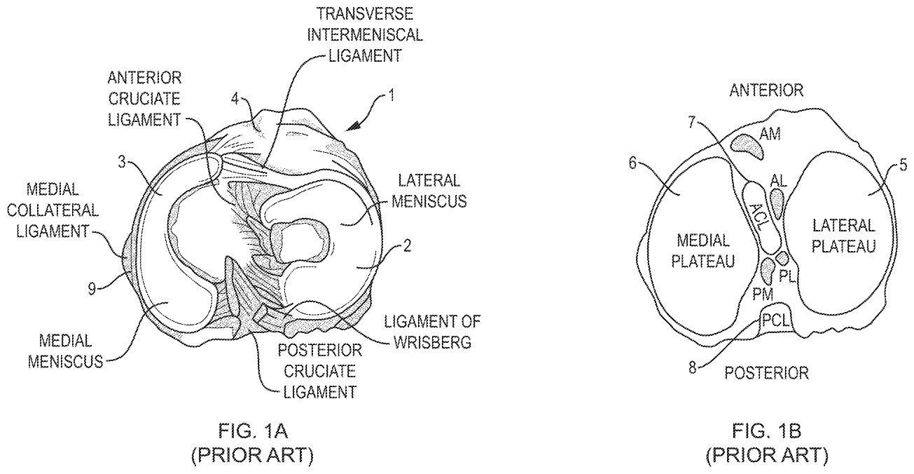

FIGS. 1A-1B are schematic illustrations of a knee 1 showing a lateral meniscus 2 and a medial meniscus 3 positioned atop a tibia 4. Each of the menisci 2, 3 is positioned on a lateral plateau 5 and medial plateau 6, respectively, to serve as a buffer between the tibia 4 and the femur 12, among other purposes. The remaining ligaments, such as the anterior cruciate ligament (ACL) 7, the posterior cruciate ligament (PCL) 8, the medial collateral ligament (MCL) 9, and other structures that support the knee 1 are illustrated at least for reference. As shown, the menisci 2, 3 account for a large portion of the surface area at the top of the tibia 4 and have a significant impact on stability of the knee 1 and its surrounding structures.

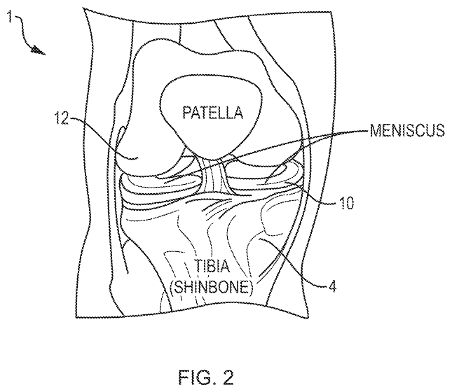

FIG. 2 illustrates an example of a meniscal implant 10 of the present embodiments being disposed within the knee 1. As shown, the implant 10 is positioned between the tibia 4 and the femur 12 to replicate a position of an anatomical meniscus to provide cushion therebetween. The implant 10 can be used as part of a meniscus repair or replacement procedure and is designed to address issues and alleviate pain associated with one or both of the preceding meniscal damage and the ensuing repair or replacement. The implant 10 is able to restore function to the space, replacing one of the menisci 2, 3, or alternatively, supplementing an injured or otherwise weakened menisci 2, 3, sometimes referred to as a partial replacement, which can occur, for example, when a majority of the meniscus is missing. Alternatively, or additionally, in some embodiments a custom implant can be designed based, at least in part, on imaging of a three-dimensional environment in the patient. For example, a shape of the implant 10 can be customized for replacing one of the menisci 2, 3 or creating a partial replacement based on a particular need of the patient.

The implant 10 can be made from a smooth material and/or include one or more features that offer stability. For example, the implant 10 can include one or more supports, e.g. struts, anchoring instruments, cushions, and/or other load or weight bearing implementations, associated therewith to enhance the strength of adjoining tissues. In some embodiments, the implant 10 can be composed of a polymer melt having a plurality of fibers that are deposited via one or more methods, as described in greater detail below, with the polymer melt having a sufficiently dense structure to be load-bearing for the adjoining bones.

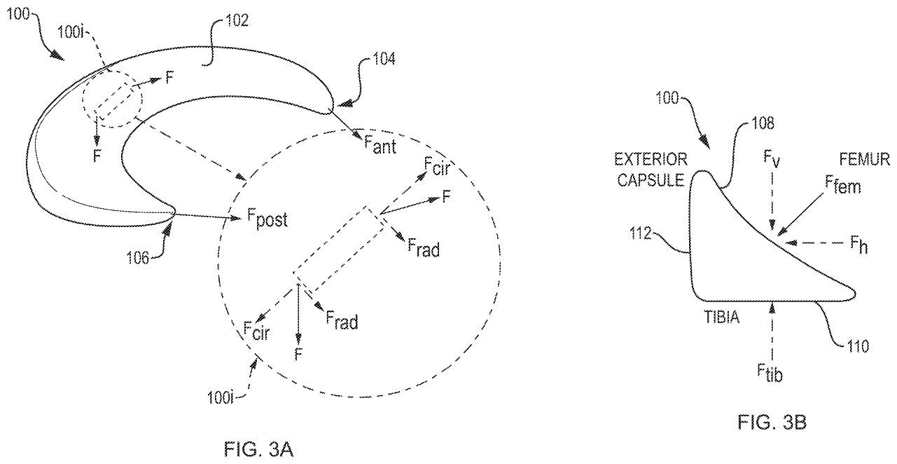

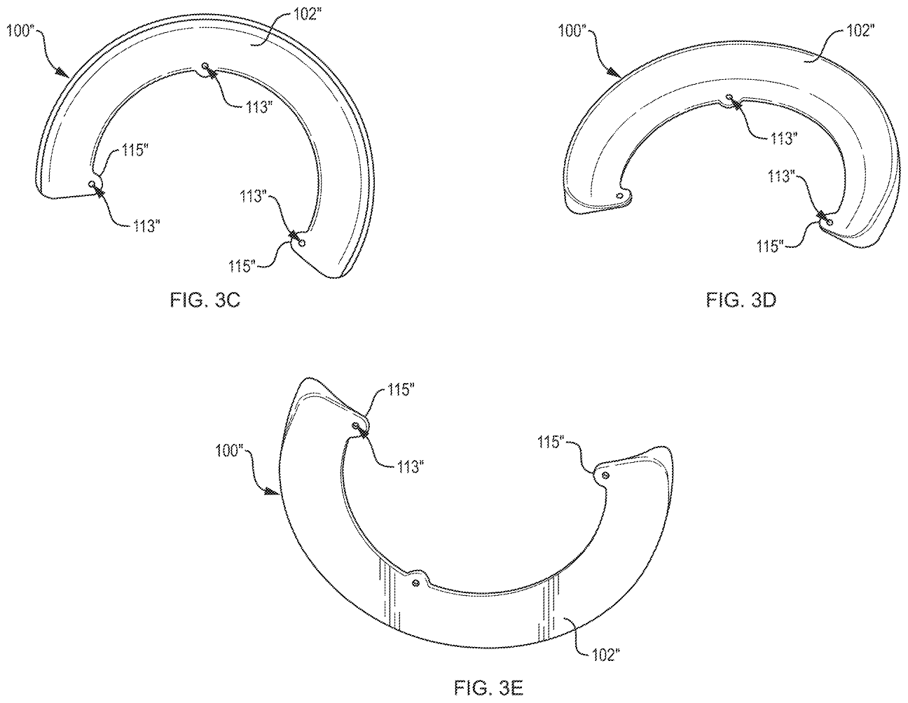

FIGS. 3A-3B are illustrations of one example of a scaffold 100 that can be used to form the meniscal implant 10. The scaffold 100 can include a body portion or body 102 disposed between a first end 104 and a second end 106. In some embodiments, the scaffold 100 can be in the shape of a continuous disk for meniscal replacement to provide tibial anchoring and serve as a cushion or pillow for the femur condyle. The continuous disk shape of the scaffold 100 can represent a semi-anatomic shape that provides superior cushion to other shapes. For example, the scaffold 100 can resemble a semi-circular shape or “C” shape, as shown, which mimics the shape of a meniscus that it is replacing to accommodate the space into which the scaffold is introduced. A person skilled in the art will recognize that the flexibility in opening and closing the “C” conformation of the scaffold 100 in such shapes can allow the scaffold 100 to be adjusted to accommodate for variance in anatomies between patients (e.g., adults, youth, etc.). A person skilled in the art will appreciate that patients are not necessarily limited to humans either, as the present disclosures can also be applied to, for example, surgical implants for other, non-human animals. In some embodiments, the scaffold 100 can be shaped to exhibit resistance to motion in lateral side-to-side movements that mimics the functionality of tissue menisci to protect the tibia 4 and femur 12 from rubbing against one another. In some embodiments, the scaffold 100 can be shaped in a manner such that the scaffold 100 is able to support a deteriorating meniscus, such shape being adjustable, conformable, and/or able to be designed to the particular support need while being based on material used and/or inflation properties. For example, in embodiments in which the scaffold 100 is prepared to custom-fit a particular patient, adjustable and/or conformable characteristics of the scaffold 100 can be molded into a desired shape for introduction into the patient.

The scaffold 100 of the present embodiments can be comprises of sterilizable porous elastomeric permeable material(s), e.g., silicone, among other materials, which can architecturally be weight and load bearing but also sufficiently porous to allow for secretion of various substances therefrom. A person skilled in the art, in view of the present disclosures, can determine the ranges anticipated to be practical for particular uses and/or preferences, e.g., weight composition, shape, and so forth of the scaffold.

The scaffold 100 can include an internal cavity that can be expandable, and thus can contract too. Alternatively, or additionally, an internal cavity can constitute space that is available within a material that is used to formulate a scaffold, like the scaffold 100″ that is created using an electrospinning process. The internal cavity can be loaded with one or more substances that can be secreted therefrom, for instance when the scaffold is stressed 100, e.g., compressed. The secretion of the material can be to an environment outside of the scaffold 100, such as a surgical site where the scaffold 100 is placed and/or anatomy surrounding the same. The secretion can be at or proximate to a surgical site. For example, the scaffold 100 can be loaded with a medicant, e.g., an analgesic, and/or hyaluronic acid (HA), to lubricate the surrounding space and provide pain management. In some embodiments, higher molecular weight acids can be preferred for the HA composition used with the scaffold 100, as discussed below. In some embodiments, the HA can be the carrier for the medicant that can be used in the implant. Additional details about secretion and/or dispersing of a substance(s) disposed in the internal cavity of the scaffold 100 is described below with respect to scaffolds having multiple chambers, though such secretion and dispersing can be applicable to single chamber scaffolds as well. One skilled in the art will recognize that the scaffold 100 can include an expandable, or deformable, external cavity, or outer shell, that can correspond to the internal cavity. The external cavity can include one or more filling openings that can receive a substance therein to inflate the external cavity. In some embodiments, the external cavity can expand to correspond to expansion of the internal cavity to avoid cracks, damage, and/or limit pressure build up with a limited outer shell resulting in no change in control volume. Moreover, it will be appreciated that an absence of the external cavity, and/or if the external cavity is rigid, can result in an absence of passive release action of substances from the scaffold 100 due, at least in part, to no load being imparted on the inner cavity.

In some embodiments, the scaffold 100 can be loaded with the one or more substances in situ, though in some embodiments, the scaffold 100 can be preloaded with the one or more substances prior to introduction into the body. The medicant can include an analgesic, a non-steroidal inflammatory drug (NSAID), and/or a non-opioid, among other substances. In some embodiments, the substances can be compartmentalized such that when two or more substances are used, e.g., analgesic and HA, the substances do not combine within the scaffold, as discussed in greater detail below.

As noted above, the scaffold 100 can be shaped to accommodate a shape of the space in which it is inserted. For example, the scaffold 100 can conform to the space between the tibia 4 and femur 12 to replicate the structure of an anatomic meniscus therebetween. Alternatively, as also noted above, in instances where the scaffold is not being used as a total meniscus replacement but instead is designed to support a deteriorating meniscus, it can be shaped to provide the support that is appropriate or necessary to allow the supported meniscus to perform as if the meniscus was not deteriorating. This may include shaping or otherwise designing the scaffold in a manner where the scaffold fills in for deteriorated portions of the meniscus and/or disposing the scaffold in a location that supports movements of the body that are inhibited due to the deterioration of the meniscus. In some embodiments, the scaffold 100 can be anchored into the tibia 4, though in some embodiments, the scaffold can be anchored to the femur 12, and/or adjoining or otherwise nearby structures, during full and partial meniscus replacements.

As shown in FIG. 3B, the scaffold 100 can include a wedge shape that anatomically accommodates the joint space. For example, the wedge shape can mimic, be akin to, or substantially akin to a shape of a meniscus. The wedge shape can allow separate surfaces of the scaffold 100 to be positioned adjacent to the tibia 4 and femur 12 for implantation. For example, a first surface 108 can be positioned adjacent to the femur 12, a second surface 110 can be positioned adjacent to the tibia 4, and a third surface can be positioned to an exterior capsule of the body. It will be appreciated that the term “positioned adjacent to” can refer to the tibia 4, femur 12, and exterior capsule abutting and/or being in contacted with each of the first, second, or third surfaces 108, 110, 112, respectively, such that the scaffold 100 can be anchored thereto.

In some embodiments, such as illustrated in FIGS. 3C-3E, a scaffold 100″ can include a plurality of bores 113″ extending through a scaffold body 102″ to assist in anchoring the scaffold 100″ at a desired surgical site. As shown, the bores 113″ can be formed in extensions 115″ that can extend radially outward from the body 102″. The bores 113″ can be configured to receive sutures, anchors, and/or other coupling devices therethrough to anchor and/or dock the scaffold 100″ to bone surfaces for implantation.

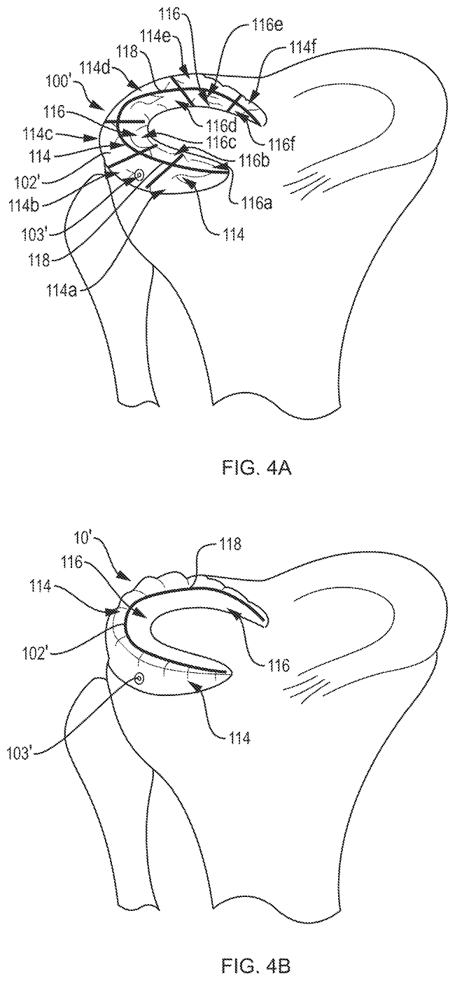

The scaffold 100 (and other scaffolds provided for herein otherwise derivable from the present disclosures), and more specifically the scaffold body 102, can have a preformed shape, such as the wedge shape, with the preformed shape including three dimensions of significance (X, Y, and Z) from the outset. Dimensions of significance means that the dimensions that define, for example, a length, width, and height of the scaffold 100 have a presence of value beyond being substantially flat. This is in contrast, for example, to the embodiment of a scaffold 100′ illustrated in FIGS. 4A-4B, in which that scaffold 100′ is inflatable such that it begins as more of a two-dimensional shape. That is, prior to inflation, the third, Z-dimension is not a dimension of significance because the scaffold 100′ in an uninflated state is substantially flat. Of course, as described herein, substances and/or materials can be added or removed from the body 102, thus causing it to change shapes and possibly even become flat. In some instances, the scaffold 100 can be designed to begin flat and inflate, a process described in greater detail with respect to the scaffold 100′ of FIGS. 4A-4B.

The scaffold 100 can include a compressive loading profile configured to withstand one or more forces exerted by adjoining anatomy that is in contact with the scaffold 100. The performance can be akin to that of fully functioning, non-deteriorated meniscus. For example, the body 102 can distribute forces in the radial and circumferential directions across the body 102 to prevent breaking, rupture, and/or other types of failure known to one skilled in the art. Instead, a force exerted on the body 102 can be distributed horizontally and/or vertically across the body towards the first end 104 and/or the second end 106, as shown, for example, with respect to the force of the femur F_{fem} in FIG. 3B.

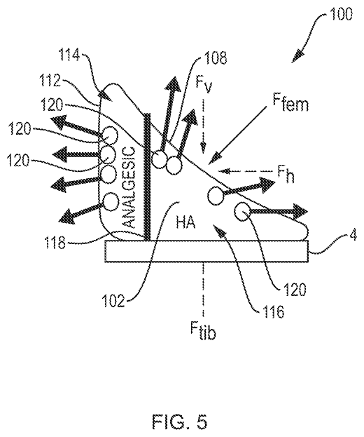

In accordance with the illustrated embodiment, once anchored, the scaffold 100 can be configured to compress in response to various forces acting on it. These forces can compress the body portion 102 and/or the ends of the scaffold to release the medicant and/or HA from the body portion 102. In some embodiments, these forces can cause the scaffold 100 to compress and conform in response to the body structures acting on it. For example, as shown in an inset image 100i of FIG. 3A, one or more forces F on the body 102 of the scaffold 100 can be broken down into circumferential forces F_{cir} and radial forces F_{rad}, thus illustrating how the forces are distributed along the body 102. The design of the scaffold 100 is such that the impact of the various illustrated forces on the body 102 is minimized. When the scaffold 100 is anchored, as shown in FIG. 3B, the body portion can be compressed by the femur and/or tibia via F_{fem} and F_{tib}, respectively, while the ends experience forces from the anterior and posterior portions of the knee, Fₐₙₜ and Fₚₒₛₜ. These compressive forces can release the medicant and/or HA contained within the internal cavity, as shown in FIG. 5 below. The compressive forces can be generated by way of the anatomy of the patient, such as portions of the body in contact with and/or adjacent to the scaffold 100. A person skilled in the art will appreciate ways by which such compressive forces can be imparted on the scaffold 100. At least because the scaffold 100 is intended to replace and/or support (in other embodiments) a meniscus, the same body forces imparted on a meniscus can be experienced by the scaffold 100. The compressive forces may include those imparted directly on the scaffold 100 by a body part, or indirectly on the scaffold 100 by way of a series of events (i.e., more than one) that creates the compressive force on the scaffold 100 in response to some movement of the body.

FIGS. 4A-4B illustrate an example of an inflatable scaffold 100′ that forms the implant 10′ when inflated. For example, as shown in FIG. 4A, the scaffold 100′ can be delivered into the space in an unfilled, or flat, state and filled in situ to anatomically accommodate the meniscal space on the medial and/or lateral plateaus. In embodiments in which a scaffold is used to support an existing, deteriorating meniscus, it can be filled in a manner that allows it to conform to the desired shape to offer the desired support for the deteriorating meniscus. Introduction in an unfilled state allows the scaffold 100′ to have a smaller profile, which can promote use in minimally invasive surgery and results in less damage to surrounding tissues during delivery. As shown, the scaffold 100′ can include an opening 103′, sometimes referred to as a filling opening, formed or otherwise disposed on a body 102′ of the scaffold 100′ to couple to one or more devices for inflating the implant. In some embodiments, the opening 103′ can receive air to inflate and/or allow substances to exit the implant to deflate, such as for removal of the scaffold 100′ from the body.

Once inflated, the scaffold 100′ can form an implant 10′ that accommodates the space, as shown in FIG. 4B. It will be appreciated that the implant 10′ in the filled state can be customized to patient anatomy or conform to the space in which it is disposed, as well as conform in other ways as described herein. For example, after inflation, the implant 10′ can resemble a meniscus and functions to reduce stresses on the adjoining bones of the knee, as discussed with respect to the implant 10 above. By way of further example, an inflatable implant can be designed to conform in view of a shape, size, and/or condition of a deteriorating meniscus to which the inflatable implant is designed to support.

Filling and/or refilling of the scaffold 100, 100′ with substances to deliver to the surgical site and/or inflate/deflate the scaffolds can occur in a number of ways. For example, in some embodiments, the scaffold 100 can be inflated and/or have substance(s) added manually once anchored in the body to transition the scaffold into the implant. Substances such as medicant and/or general medical grade lubricants, e.g., HA, can be added into the scaffold 100, though, in some embodiments, mechanical components, such as mechanical dilators (e.g., shims) can be inserted to change a shape of the scaffold 100. In such embodiments, the scaffold 100 can include a free-form design that can compress, but has a natural resting state that can naturally conform and expand to the geometry, though the HA/lubricant/medicant may undergo passive release and depletion cycles. Alternatively, or additionally, the scaffold 100 can be considered a bladder, inflated by adding a medical grade fluid, such as saline, to fill a non-permeable portion thereof. While filling and/or refilling of the scaffold is discussed with respect to the scaffold embodiment of FIG. 3A, a person skilled in the art will recognize that one or more of these techniques can be used for the scaffold 100′ of FIG. 4A.

In some embodiments, the scaffold can be filled passively with a dry, larger molecular weight feasible HA that can absorb water once implanted. A person skilled in the art, in view of the present disclosures, can determine the ranges anticipated to be practical for particular uses and/or preferences. After implantation, these compounds can expand and fill the scaffold 100 until it is sufficiently inflated to accommodate the space.

In some embodiments, the scaffold 100 can be designed such that different regions of the scaffold 100 included different compounds to form an implant 10 having multiple compounds contained in the scaffold 100 (or multiple substances if one or more of the compounds is a single substance. As shown in FIG. 5, the analgesic and the HA can be loaded separately into designated and/or designed regions of the scaffold 100 to allow release of substances in desired, different directions and/or locations. For example, the analgesic and the HA can be loaded into the scaffold 100 such that the analgesic can extrude, secrete, and/or elute into an external capsule space when compressed and/or a load is applied thereto, while the HA can extrude, secrete, and/or elute into the synovial space. It will be appreciated that the direction in which the compounds are extruded, secreted and/or eluted can depend on a position of the implant 10 within the disc space and the portion of the implant 10 in which the compounds are loaded.

The present disclosure, and a person skilled in the art in view of the present disclosures, will understand a variety of ways by which the scaffold 100 can be designed to allow for different substances and/or compounds to release at different times, in response to different conditions, and/or in different locations and/or directions. In some embodiments, the scaffold 100 can include a plurality of chambers formed therein for receiving compounds in these designated and/or designed regions of the scaffold 100. For example, substances introduced into the scaffold 100 can be compartmentalized within the internal cavity such that when two or more substances are used, the substances stay separated within the implant 100. As shown in FIGS. 4A, 4B, and 5, the implant 100 can include a plurality of chambers 114, 116 that can be separated, for example, by one or more dividers 118. In some embodiments, the first chamber 114 can be an outer chamber that can include an analgesic disposed in it, while the second chamber 116 can be an inner chamber that can include HA disposed in it, though it will be appreciated that the location of the analgesic and the HA can be switched between chambers. Still further, each of the outer and inner chamber 114, 116 can be further subdivided into smaller chambers, with each chamber being able to release materials therefrom independent of the other chambers, though certainly multiple chambers can be configured to release simultaneously as desired. In the illustrated embodiment, as shown in FIG. 4A, the outer chamber 114 includes outer sub-chambers 114a, 114b, 114c, 114d, 114e, and 114f and the inner chamber 116 includes inner sub-chambers 116a, 116b, 116c, 116d, 116e, 116f. It will be appreciated that the divider 118 can be impermeable to either or both of the analgesic and the HA, though, in some embodiments, the divider can include one or more pores therein to allow permeation of substances between the chambers 114, 116. Moreover, while the scaffold 100 is shown having two chambers, implants having three or more chambers can be contemplated by one skilled in the art in view of the present disclosure. It will also be appreciated that, in some embodiments, the scaffold 100 can include compartments without a divider, with each compartment being defined within the body of the scaffold 100 to fill to a specific size during implantation.

In some embodiments, the scaffold 100 can include one or more openings 120, sometimes referred to as dispersing openings, that open when the implant 10 is acted upon by a force and/or subjected to a certain pressure threshold. For example, in the absence of a compressive force on the implant, the openings 120 can be biased closed to retain the shape of the implant. In response to a compressive force, the openings 120 can open to release the medicant, HA, and/or other material contained in the scaffold 100. Each chamber 114, 116 can include a plurality of openings 120 to release contents of the chambers in various directions, as shown, though in some embodiments, each chamber 114, 116 can include a single opening in each chamber. Alternatively, or additionally, in some embodiments the implant 10 can include an outer surface that is porous to allow the medicant, the HA and/or other material to be released through a designated portion of a surface area of the implant 10 that is porous. For example, a force F_{fem} of the femur 12 onto the first surface 108 of the body 102 can release the medicant or HA contained therein. With axial loading occurring superior and inferior to the implant, the HA can be placed in regions that need large amounts of lubrication. As shown, F_{fem} can release HA out of areas of high friction, e.g., the first surface 108, towards the femur 12. In some embodiments, the medicant can be substantially simultaneously released out of the third surface 112 towards the exterior capsule, as the medicant is non-specific in region that can be aimed to diffuse into the local environment. Moreover, in some embodiments, an amount of the medicant that can be released from the body 102 can be proportional to an amount of compressive force exerted thereon.

In some embodiments, the opening can include a float valve on a sub pump to regulate an amount of medicant or HA that is released. Additionally and/or alternatively, in some embodiments, control of release can be performed mechanically with polymer degradation over time. For example, release can be based, at least in part, on structural tear down where compression causes chambers to break and/or open, thus releasing the HA, and/or time-released decay of polymers over time can be based, at least in part, on their reaction to the biological environment. Alternatively, or additionally, the implant 10 can include capillary tubes to limit the volume of medicant or HA being released. The capillary action can be based, for example, on pressure gradients and can allow for not only release of the material that is inside the implant at the start, but also the ability to semi-absorb some of the contents that were released for potential re-use. The capillary action can be controlled via the permeable membrane that can be designed to allow HA and/or the lubricant back into the implant 10 (similar to a sponge) based, at least in part, on size, polarity, and/or charge, and so forth, when subjected to the release of the compression/pressure.

In some embodiments, the implant 10 can be associated with a control system (not shown) configured to detect pressure and/or HA concentration in the implant. For example, the control system can be associated with a UV specification device that can scan the implant 10 to determine a volume of HA contained in the implant 10. These volume measurements can be used to regulate pressure within the implant and prevent overfilling, which could cause the implant 10 to rupture and possibly damage surrounding tissues and the like. For example, the control system can issue a signal to indicate to a user, e.g., a patient or physician, to manually adjust the flow rate and/or pressure of the medicant or HA flowing into and/or out of the implant. In some embodiments, the control system can be in communication with the pump, e.g., pump 230 as discussed below, to automatically trigger increase and/or decrease of flow of the HA or medicant to the implant in real-time. It will be appreciated that references to HA and/or medicant, both above and throughout the specification, can be applicable to any material, medicant, substance, compound, and so forth, disposed in the scaffold in conjunction with and/or in view of the present disclosures. The control loop provided for by the present disclosures allows various parameters to be monitored and the outflow of medicant and/or HA to be controlled. In some embodiments, diagnostic data gathering of various compression pressure gradients experienced by the body portion 102 of the scaffold 100 can use this data to evaluate future potential procedures to be performed on the target area or for monitoring of additional wear and/or over-compression over time can be performed. By way of non-limiting example, in some embodiments, the scaffold 100 can be associated with a Flexible Force Sensitive Resistor (FSR) that can be lined within a protected surface of the body 102 that can experience heavy friction/compression, e.g., the first surface 108 that abuts the femur. The force/pressure can be quantified by the sensor to gather information for a physician to understand the wear and tear on the implant and/or inner channels can be opened based on the reading on the sensor (or other sensing elements incorporated into the scaffold 100 and/or the surrounding environment) to allow for data collection. In some embodiments, sensors can be positioned relative to one another at different calibration states to ensure tracking of stressed and unstressed states. These sensors can provide information to the control loop. The sensors can be incorporated as part of the scaffold 100 and/or can be positioned and/or placed to obtain relevant data from a surgical site and/or from the patent more generally and/or from an environment more generally to the extent such measured factors may impact how data is gathered and/or monitoring is performed.

The release of the HA or medicant from the implant 10 can be passive, substantially continuous, responsive to certain conditions, e.g., force or impact on the body, use of joint, and so forth, and/or time targeted. It will be appreciated that, for the purposes of this disclosure, substantially continuous can refer to a release of HA or medicant from the implant 10 for approximately 90%, approximately 95%, or approximately 99% of the time that the implant is anchored in the body. Time targeted release, which can generally be viewed as a passive approach, can refer to degradation of one or more of the HA and/or medicant from the implant 10. For example, in the implant 10 of FIG. 5, the scaffold 100 can exhibit slow decay while the HA and/or the medicant can exhibit fast decay from the implant. In some embodiments, the time targeted release can be configured to deplete the reservoir of the HA and/or the medicant from the implant 10 quickly following implantation of the implant to promote healing after the procedure. In some embodiments, time targeted release from the implant 10 can occur when the implant is crushed, broken, and/or squeezed to release certain aspects contained therein.

Release of the contents from the implant 10 can occur in a variety of ways. As noted above, in some embodiments the HA can be released at high contact stress surfaces from the body 102 of the scaffold 100 in response to a compressive force. Release of HA can occur as needed, though in some embodiments, can be automated to release a certain amount of times per a period of time, e.g., an hour, a day, a week, a month, a year, and so forth. The automated release can allow for routine upkeep of the implant 10 without manual interference, ensuring that the body is sufficiently lubricated with respect to adjoining tissues to reduce risk of failure. Alternatively, or additionally, the implant 10 can include a spring-loaded release that allows HA to flow into the joint space when a spring is compressed by a force, e.g., F_{fem}. In some embodiments, release of the medicant can occur during off-axis loading, which can stimulate analgesic to be eluted from the implant.

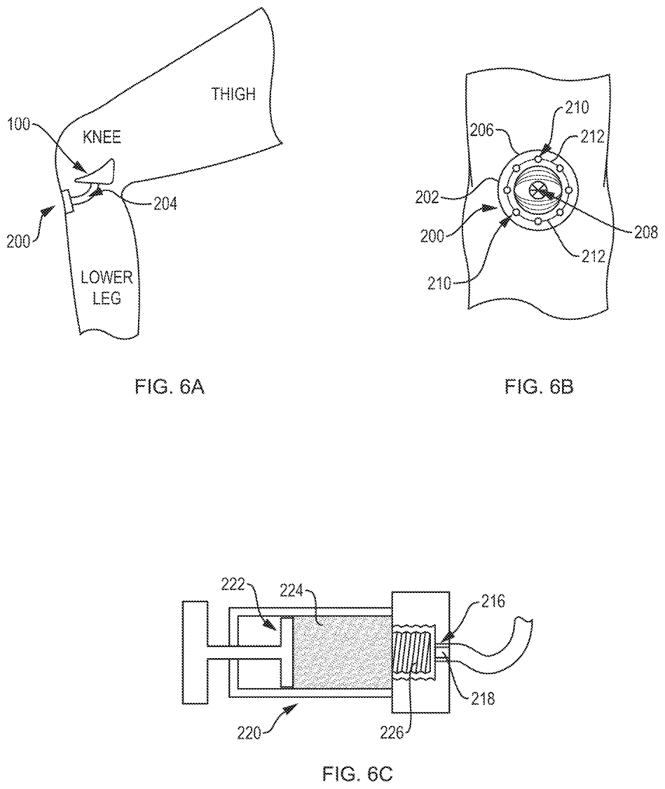

In some embodiments, the implant 10 can include an access port 200 formed on or otherwise associated with the implant 10 to facilitate extrusion, secretion, and/or elution of substances from the implant 10. For example, the implant 10 can include a vascular access port 200 having a pressurized reserve that opens under a certain pressure threshold to aid in the release of the medicant and/or HA, and is otherwise closed. In some embodiments, the access port 200 can be engineered into the meniscus to allow ease of access for refilling thereof. For example, the access port 200 can be coupled to the implant such that the port 200 is implanted on a lateral compartment of the leg. FIGS. 6A-6C illustrate an exemplary embodiment of such an access port 200 that can be used with the implant 10 of the present embodiments to facilitate filling and/or refilling of the scaffold 100 with medicant and/or HA. As shown, the access port 200 can include an implantable interface 202 and a tube or catheter 204 can allow for fluid communication between the access port 200 and the scaffold 100. The tube 204 can be part of the access port 200, part of the scaffold 100, or it can be its own separate component that can be selectively coupled to the access port 200 and scaffold 100. The implantable interface 202 of the access port 200 can be secured to the skin surface to provide access to the scaffold 100 via minimally invasive means once the scaffold 100 is implanted, using techniques for such securement known to those skilled in the art. Substances introduced through the access port 200 can flow through the tube 204 and into the scaffold 100 to manipulate an orientation of the implant 10 without dislodging the implant from the space.

FIG. 6B illustrates the access port 200 in greater detail. As shown, the implantable interface 202 can include a body 206 having a central opening 208. The central opening 208 can connect to the tube 204 that leads to the scaffold 100 to allow for fluid communication between the two such that substances can pass therebetween. The central opening 208 can include a hemostatic valve 216 (see FIG. 6C), for example to reduce blood loss and risk of air embolism. The body 206 can further include a series of holes 210 formed on its periphery. The holes 210 can be configured to secure the access port 200 to the skin surface. As shown, the holes 210 can include a series of sutures 212 strung therethrough to hold the access port 200 flush with the skin. A person skilled in the art will recognize that alternate methods for attaching the access port 200 to the skin can include dermabond, glue, threading, and so forth. It will be appreciated that while the access port 200 is round, other embodiments of the access port can be shaped as ovals, triangles, squares, rectangles, and so forth.

In some embodiments, the port 200 can be filled with a needle or catheter to allow for refilling the implant with the HA or medicant via manual distribution. FIG. 6C illustrates an exemplary embodiment of a syringe 220 that can be used with the access port 200. As shown, the syringe 220 can include a reservoir for storing the medicant or HA 224 and a threaded dispenser 226. The threaded dispenser 226 can include threads that can mate with one or more internal threads of the central opening 208 on the access port 200. Once mated such that the threaded connection of the dispenser 226 and the central opening 208 are sealed, a nozzle 218 of the syringe 200 can open the hemostatic valve 216 to deliver the contents of the reservoir 222 through the central opening 208.

In some embodiments, a volume of the syringe can be about 10 cubic centimeters (cc). Moreover, in some embodiments, the HA or medicant 224 can be initially injected as a large volume bolus and then maintained with an additive cc volume introduced per day, week, month, year, or any time period.

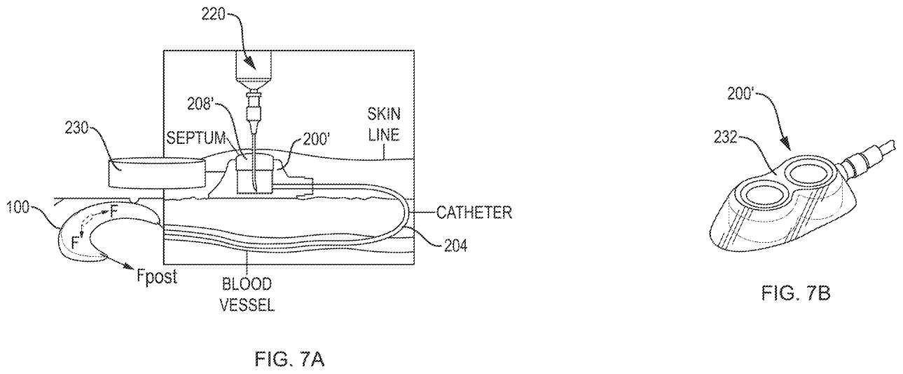

As noted above, in some embodiments, the analgesic or HA can be filled and/or refilled using a pump 230, as shown in FIGS. 7A-7B. While the pump is shown in FIGS. 7A-7B, it will be appreciated that the pump 230 can be used in conjunction with the access port of FIGS. 6A-6C. The pump 230 can be in fluid communication with the access port 200 and/or the scaffold 100 via a manual connection that forces compounds that enter through the access port 200 via the central opening 208 to flow to the scaffold 200 through the tube and/or catheter 204, as described above. The pump 230 can be used to control flow rate and/or pressurization of the implant 10 by regulating a concentration of the HA and medicant 224 contained in the scaffold 100 and flowing thereto. For example, the pump 230 can define a direction of the fluid flow through the access port 200 to facilitate flow that resembles a one-way valve towards the scaffold 100.

In some embodiments, the pump 230 can be used in combination with a vascular access port and/or an Implanted Central Venous Access Port (IVAP), which are known to those skilled in the art. Moreover, as shown in FIGS. 7A-7B, in some embodiments, an access port 200′ associated with the pump 230 can include a septum 232 disposed proximal to the central opening 208′ thereof. As shown in FIG. 7B, the septum 232 can include a film disposed over the central opening 208 to allow the syringe 220 to be inserted therethrough. The access port 200′ can be about 2.54 cm long and the septum 232 can have a length approximately in a range of about 9 millimeters to about 10 millimeters.

The pump 230 can be elongated to facilitate implantation with minimal interference to surrounding structures. In some embodiments, the pump 230 can further include polymer wings (not shown) on it to promote stabilization. The pump 230 can be implanted in a lateral compartment of the leg in proximity to the access port 200′, as shown. In some embodiments, the pump 230 can be stitched to the skin initially to stabilize and promote adherence. When implanted, the pump 230 can adhere to the fascia in about a week, which can allow it to remain secure to the body of the patient.

The pump 230 can be filled with a gas, e.g., Freon gas, to keep a steady pressure in the pump, which can promote a fixed rate flow of medicant or HA to the scaffold 100. The HA can be capillary fed into the scaffold 100, with the HA seeping out of the scaffold in response to a compressive force. In some embodiments, the pump 230 can include micropores that form a compression pressure gradient to ensure flow of substances in a given direction. It will be appreciated that the pump 230 can use hydrophobicity to control compression pressure gradient and flow rate. As noted herein, controlling compression pressure gradient and/or flow rate, among other properties, can control capabilities of the scaffold 100, such as the amount and speed at which substance(s) flow from the scaffold 100. The pump 230 can also be associated with the control system of the present embodiments, as discussed above, to actively monitor pressure/concentration and responsively pump HA and analgesic into the joint space, though, in some embodiments, manual activation of the pump 230 can be used to accomplish such monitoring.

Contents delivered through the central opening 208′ can flow through the flexible tube 204 to the scaffold 100 to fill and/or inflate the scaffold 100. In some embodiments, a plurality of tubes can be connected between the central opening 208′ and various portions of the scaffold 100 to deliver one or more specific substances to designated chambers of the scaffold body, such as to the medicant and the HA being delivered to chambers 114, 116 discussed with respect to FIG. 5 above.

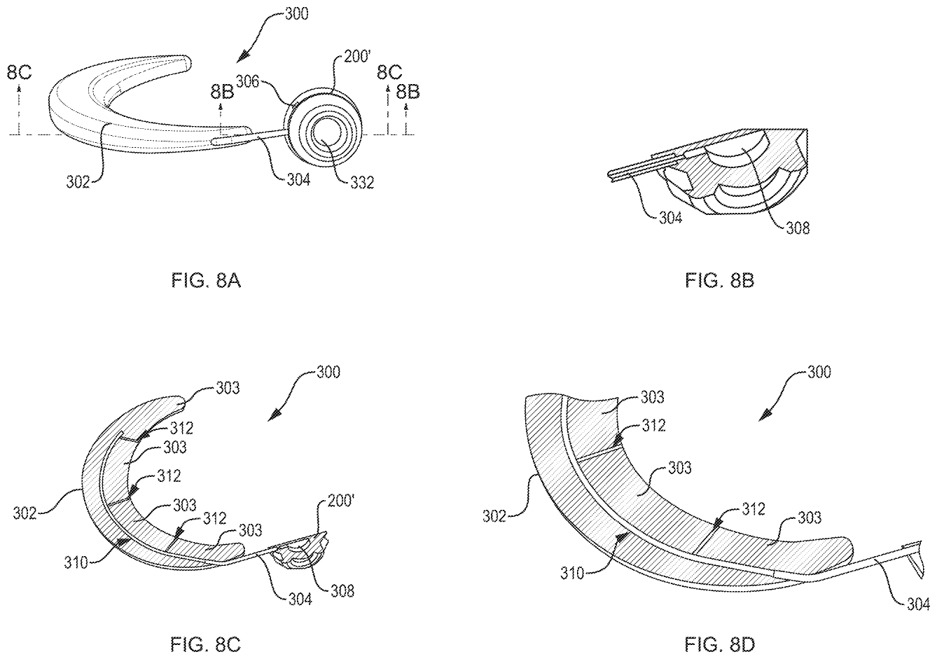

FIGS. 8A-8D illustrate another embodiment of a scaffold 300 having a body 302 that includes a plurality of port chambers 303 therein. As shown, the scaffold 300 can be connected to an access port 200′ by one or more tubes 304. The access port 200′ can include a plurality of openings or slots 306 that can be configured to receive the tube 304 for forming a fluid connection between the access port 200′ and the scaffold 300. As shown in FIG. 8B, the openings 306 can be in fluid communication with the central opening 308 of the access port 200′ to pass substances through the tubes 304 to the scaffold 300. In some embodiments, substances can pass through a septum 332 to pass through the central opening 308 and flow to the scaffold 300 via the tube 304.

FIGS. 8C-8D illustrate the connection between the tube 304 and the scaffold 300. The scaffold 300 can include a channel 310 formed in the body 302 that distributes substances flowing through the tube 304 throughout the scaffold 300. As shown above, the scaffold 300 can be divided into chambers 303, with each chamber 303 being defined between branched pathways 312 that are in fluid communication with the channel 310. The branched pathways 312 can receive the substances flowing through the channel 310 to separate the substances within the scaffold 300, thereby compartmentalizing these substances. Flow of medicant/HA through the branched pathways 312 can be based, at least in part, on permeable membranes and/or capillary-like passages to the outer surface. In some embodiments, porous coatings can be used to facilitate flow. For example, medicant/HA can flow through the channel 310 to fill the chamber 303, at which point the substances can interact with the permeable portion of the scaffold 300.

In some embodiments, the scaffold 100 can be manufactured via electrospinning, including but not limited to in situ. Electrospinning is a fiber production method that can uses electric force to draw charged threads of polymer solutions or polymer melts. In electrospinning, a flow rate can electrically charge jets of fluid that exit the device. Electrospinning is a feasible and versatile technique that can be used to produce nano- to micro-scale fibers that mimic the microarchitecture of native extracellular matrix and is an effective approach to prepare nanofibrous scaffolds for constructing engineered menisci. Electrospun scaffolds can be capable of inducing colonization of meniscus cells by modulating local extracellular density and stimulating endogenous regeneration by driving reprogramming of meniscus wound microenvironment. In some embodiments, electrospinning can create a randomized pattern of filament spinning that can create instabilities that harden and become an implant.

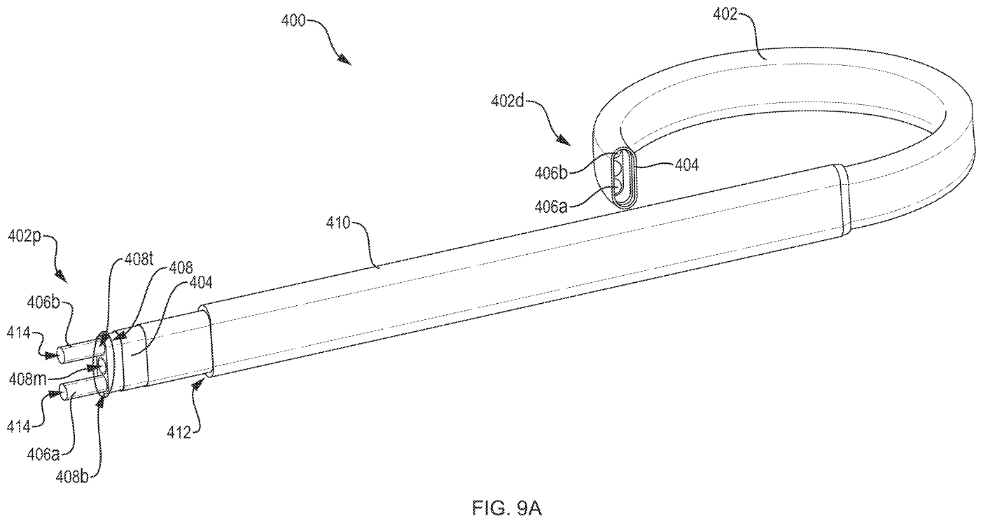

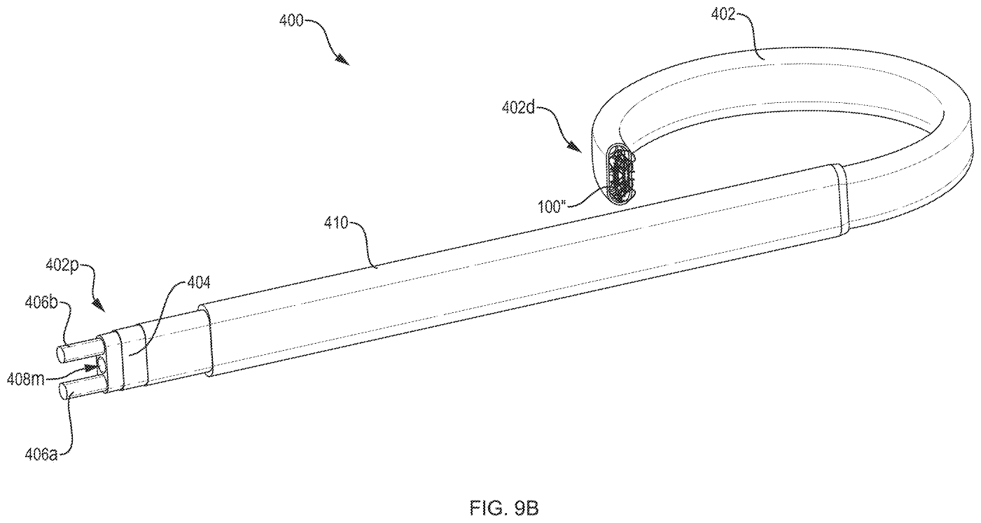

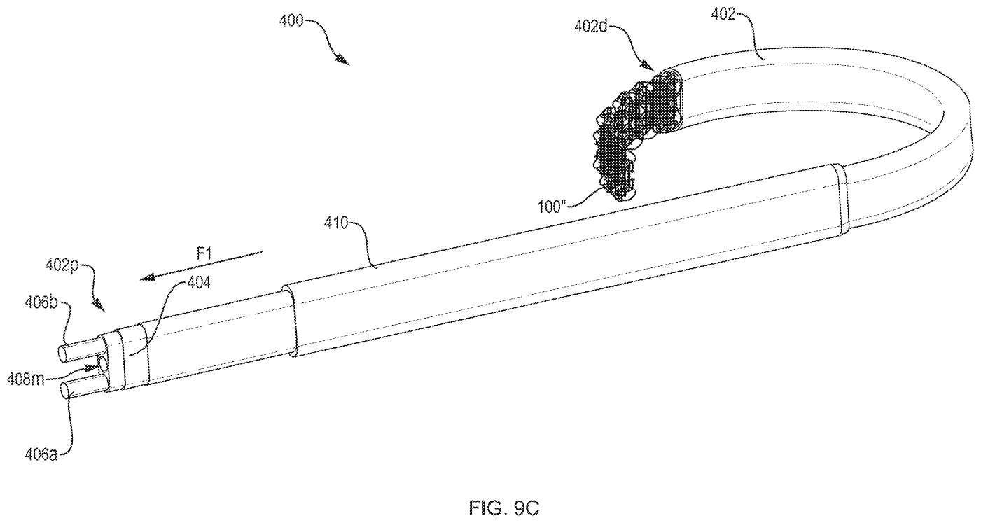

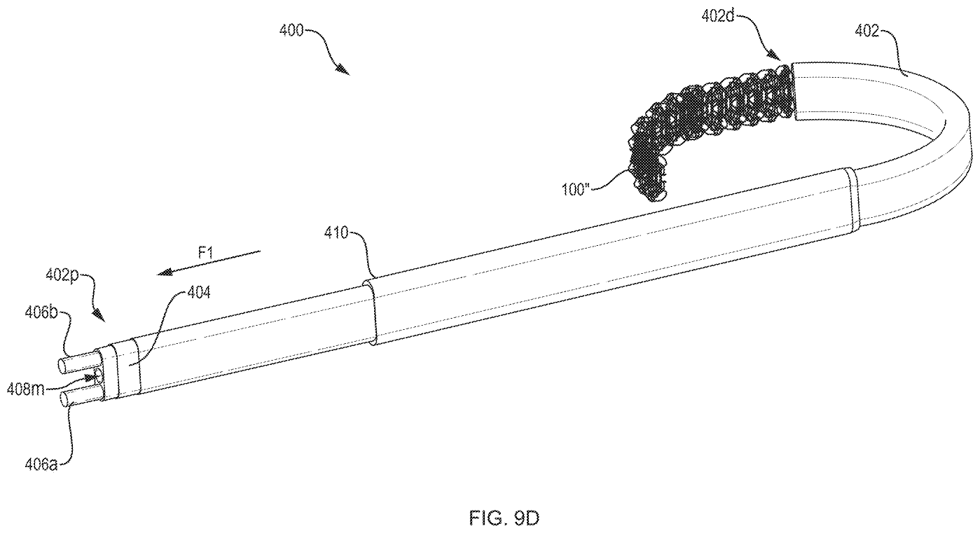

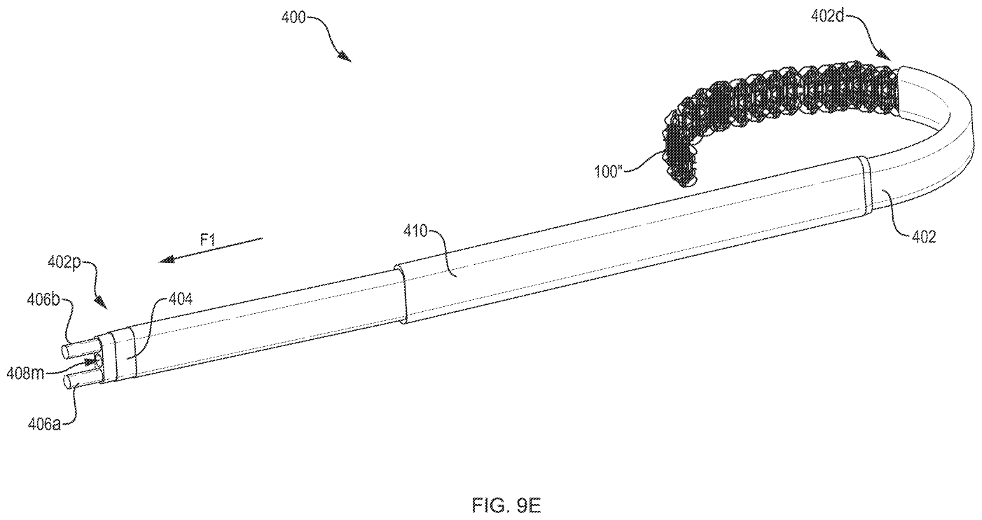

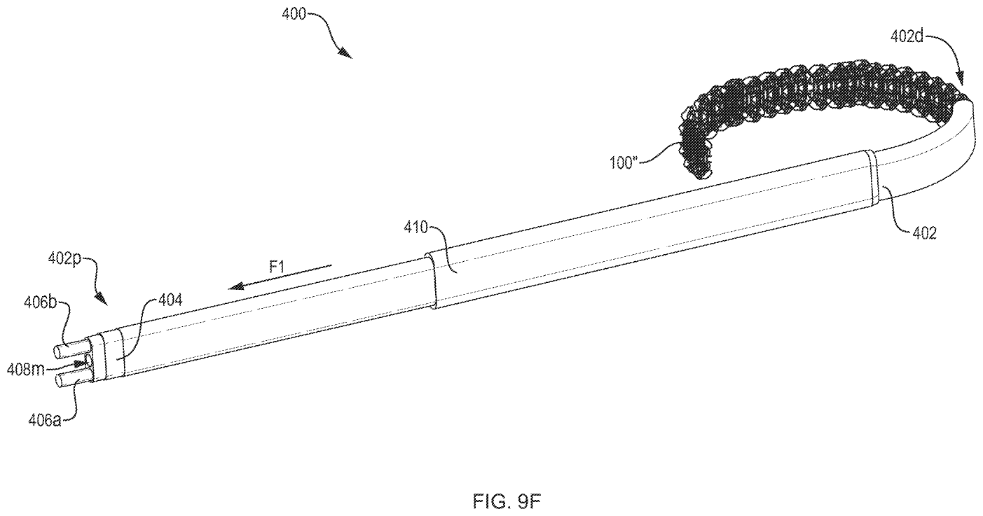

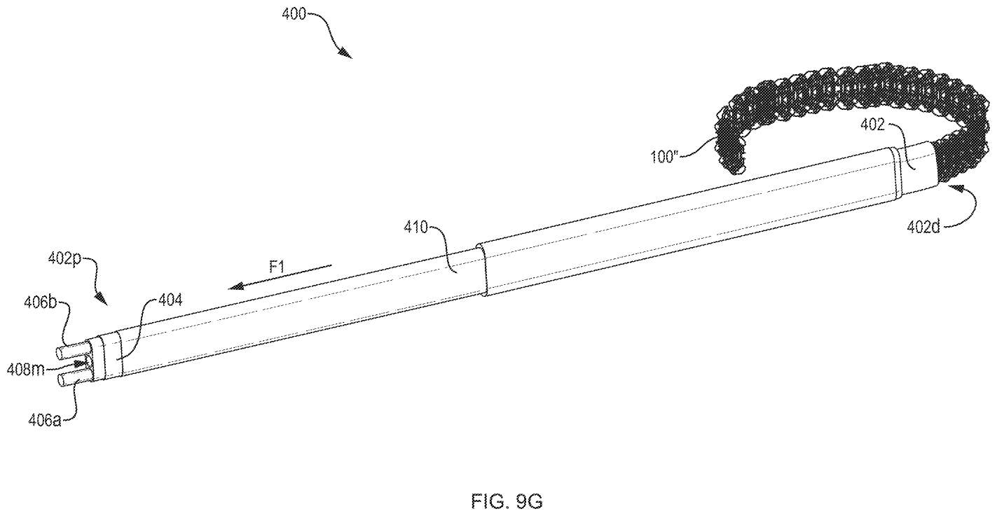

FIGS. 9A-9G show an electrospinning device 400 that can be used to create the implants of the present embodiments. As shown, the electrospinning device 400 can include a body 402 that extends from an introductory or proximal end 402p to a drawing or distal end 402d. The electrospinning device 400 can include a cannula 410 having a channel 412 therein through which the body 402 of the electrospinning device 400 passes. The body 402 can be made from one or more flexible materials such that a portion of the drawing end 402d that is disposed outside the cannula 410 can be biased, as shown by way of example, to curve inwardly to form a substantially round distal end. Some non-limiting examples of materials that can form the body 402 can nitinol and other shape memory alloys. As shown, the drawing end 402d can curve to an extent such that, when fully inserted, a terminal end thereof can be disposed proximate to an intermediate portion of the body. When translating the body 402 through the cannula 410, the cannula 410 can counteract the bias of the body 402 such that the drawing end 402d straightens out to pass through the cannula 410.

The body 402 can include one or more collector electrodes 404, one or more charge electrodes or spinnerets 406a, 406b, and a series of lumens 408t, 408m, 408b (collectively 408) formed therein. As shown, the body 402 can include the collector electrode 404 disposed along the body proximate to each of the introductory end 402p and the drawing end 402d of the body 402. The collector electrode 404 can include a conductive substrate, such as a metal, that can function as the ground electrode and can help form a stable electric field in the spinnerets 406a, 406b. One or more tubes, for example the charge electrodes or spinnerets 406a, 406b, can pass through the series of lumens 408 to flow air and other materials from the introductory end 402p to the drawing end 402d. Using a plurality of lumens 408 can provide options for using more than one material and two unique filament structures, i.e., density. As shown, the top and bottom lumens 408t, 408b can be configured to receive the charge electrodes 406a, 406b therethrough, respectively, while the middle lumen 408m can be configured to receive return airflow therethrough. Each charge electrode 406a, 406b can include a lumen 414 extending therethrough that permits passage of air flow and/or materials. The electrospinning device 400 can be introduced through the cannula 410 into the patient's body along a path via which the device can produce the implant. For example, as shown, the drawing end 402d can curve around body structures, as discussed above, to resemble a curved shape of a meniscus spun by the device 400 as it travels along the path.

In some embodiments, the electrospinning device 400 can be a hollow, dual lumen pre-formed nitinol electrospinning catheter, as shown. The dual lumen pre-formed nitinol electrospinning catheter can be fully inserted into the joint space and the desired polymer can be load in preparation for electrospinning. Once started, the electrospinning process involves the dual lumen catheter being slowly extracted from the joint space leaving behind the desired filament structure that forms the implant. Use of the electrospinning device is discussed in greater detail below.

FIG. 9B illustrates the electrospinning device 400 beginning to produce an implant 10″ of the present embodiments via electrospinning. As shown in FIG. 9B, the electrospinning device 400 can be actuated to draw charged fibers of polymer melts. Fiber diameters of the polymer melts that are drawn up can be approximately in a range of about 50 nm to about 1,000 nm, about 100 nm to about 700 nm, about 200 nm to about 500 nm, and/or about 300 nm to about 400 nm. Some non-limiting examples of polymers that can be used for electrospinning the implant 10″ can include one or more of PCL Nanofiber, polyurethane, poly L-lactide-co-e-caprolactone (PLCL), polyglycolic acid (PGA), poly(lactic-co-glycolic acid (PLG), and other polymers known to one skilled in the art. Upon actuation, the drawing end can begin to deposit the fibers therefrom that will create the meniscal scaffold 100″. Temperature, diameter of the electrodes 406a, 406b (also referred to as a diameter of nozzles or tubes), and/or flow rate can determine the rate at which the scaffold 100″ is being created. The present disclosure allows for users, including but not limited to clinicians, to adjust factors such as the temperature, delivery component diameters (e.g., electrode, nozzle, tube, etc.) to help control an amount of medicant, HA, and/or other material(s)/substance(s) being delivered to or proximate to a surgical site. Moreover, arrangement of the fibers to form the scaffold 100″ can be determined by Rayleigh instability, which can depend on the viscosity and type of polymer being used. Instability of the fiber can cause the fibers to “spring” in space due to their inability to maintain their weight as they extrude out further into a gravity-based environment towards a charged collector plate. In some embodiments, different profiles of the fibers can be created based, at least in part, on one or more of temperature, speed, polymer, and/or ejection diameter, and so forth.

FIGS. 9C-9G illustrate the body of the electrospinning device 400 being retracted relative to the cannula 410. Retraction of the body 402 can occur by exerting a proximal force F1 onto the body 402 while keeping the cannula 410 still within the joint space. As shown, during retraction, the drawing end can deposit fibers throughout its motion. The bias of the drawing end 402d can be counteracted such that it straightens out as the body 402 is retracted when exiting the cannula 410. The fibers that are deposited can be shaped like a meniscus that form the scaffold 100″, or in other known desired shapes, such as instances where the material being deposited is shaped to support a deteriorating meniscus rather than to fully replace a meniscus. As noted above, in embodiments like the scaffold 100″, an internal cavity of the scaffold 100″ can be space that exists within or between the deposited fibers such that the substance(s) to be released (e.g., medicant, HA, etc.) as designed is located within the confines of the scaffold 100″, and thus the internal cavity of the same is more generally an internal volume of the scaffold 100″ in which such substance(s) can be disposed.

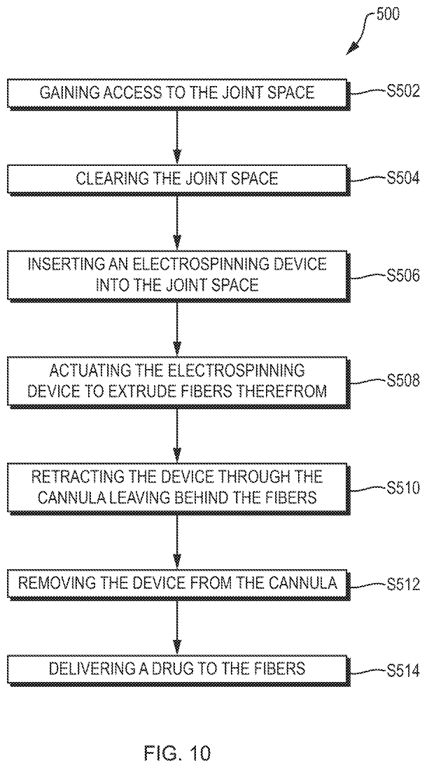

FIG. 10 illustrates an exemplary method 500 for electrospinning the scaffold 100″. As shown, the method can include gaining access to the joint space (S502). Initially, access to the joint space, e.g., the medial and/or lateral plateaus of the knee, can be gained by devices and techniques known to one skilled in the art. For example, a person skilled in the art will recognize that access to the join space can be gained using one or more of access ports, cannulas, and so forth. Once access is achieved, the space can be cleared by removing the damaged or diseased meniscus (S504), in instances where a meniscus is being replaced, or the meniscus can be prepared, if necessary, to receive supporting material in instances where the deteriorating meniscus is not being fully replaced. Other anatomies at the surgical site may also be prepared, as understood by those skilled in the art, as appropriate for the procedure being performed.

In some embodiments, the access port 200 or cannula 410 can be used to aid in the removal and creation of the new meniscus. For example, a hollow nitinol pre-formed catheter can be inserted through the cannula 410 to clear the damaged or diseased meniscus space in preparation for the new meniscus to be created. After the space is cleared, the electrospinning device 400 can be inserted into the joint space (S506). Once inserted to a desired configuration, the electrospinning device 400 can be actuated to extrude the polymer fibers therefrom (S508). Actuation of the device 400 can be performed via techniques and methods known to one skilled in the art. As the fibers are extruded, the body 402 is retracted through the cannula 410, while leaving behind the extruded fibers (S510). It will be appreciated that the plurality of lumens 408 in the body 402 can allow for extrusion of a plurality of materials that results in unique filament structures. Retraction of the device 400 continues proximally until the device 400 is removed from the cannula 410 while the cannula 400 remains in place (S512).

In some embodiments, a desired lubricating fluid and/or pain drug, e.g., medicant and/or HA, can be delivered into the filament structure (S514). For example, a pre-formed nitinol needle can be inserted through the cannula after the device is removed to inject one or more compounds to the filament of the scaffold 100″. The filament structure can be porous to allow the compounds to slowly be secreted over time. In some embodiments, the compounds can be added through a drug port. For example, a fill tube can be inserted into the filament structure and bonded thereto prior to removing the port 200 or cannula 410. Once the port 200 or cannula 410 is removed, the fill tube can be subcutaneously facilitate drug delivery into the patient.

One of the many benefits of the implants provided for herein (e.g., the implants 10, 10′, 10″) is they allow for the ability to refill the implant with medicant and/or HA, which in turn enables the implants to remain anchored within the joint space without being disturbed and without needing more traumatic surgical interventions to replace used implants while also promoting healing. As a result, the implants provided for herein can be used for a long duration of time without requiring replacement. For example, the implants disclosed herein can be considered long-term implants such that they can be disposed at a surgical site for at least one year, at least two years, at least three years, at least four years, at least five years, or longer without requiring replacement. Moreover, the implants disclosed herein can provide continuous pain management and be reactive to the environment to provide a patient-specific lubrication volume throughout the lifecycle thereof.

Examples of the above-described embodiments can include the following:

1. A surgical method, comprising:
accessing a joint space of a patient that comprises, or previously comprised, a meniscus;
delivering a scaffold to one of replace the meniscus or support the meniscus, the scaffold comprising an internal cavity, and the scaffold having one or more substances disposed in a permeable portion of the internal cavity at least one of during or after it is delivered to the joint space; and
anchoring the scaffold to the tibia,
wherein the scaffold is configured to release the one or more substances from the internal cavity of the scaffold, to the joint space, in response to one or more compressive forces exerted onto the scaffold.
   2. The method of example 1, wherein the internal cavity comprises the permeable portion and a non-permeable portion.
   3. The method of example 2, further comprising:
loading the permeable portion of the internal cavity of the scaffold with the one or more substances.
   4. The method of example 3, wherein loading the permeable portion of the internal cavity of the scaffold with the one or more substances occurs in situ.
   5. The method of any of examples 1 to 4, further comprising delivering the one or more substances to the internal cavity after the scaffold is anchored to the tibia.
   6. The method of any of examples 1 to 5, further comprising delivering the scaffold into the joint space in an uninflated state.
   7. The method of example 6, further comprising inflating the scaffold to form the implant that accommodates the joint space.
   8. The method of any of examples 1 to 7, further comprising coupling an access port to a location on the patient that is remote from the joint space, the access port being in fluid communication with the scaffold to deliver the one or more substances thereto.
   9. The method of any of examples 1 to 8, wherein delivering a scaffold further comprises electrospinning the scaffold.
   10. The method of example 9, wherein electrospinning the scaffold further comprises operating an electrospinning device to deliver material that forms the scaffold to the joint space.
   11. The method of any of examples 1 to 10, further comprising controlling at least one of a temperature of a material used to form the scaffold, a diameter of a nozzle that delivers a material used to form the scaffold, or a flow rate of a material used to form the scaffold.
   12. The method of any of examples 1 to 11, wherein the scaffold is configured to release the one or more substances substantially continuously.
   13. The method of any of examples 1 to 12, wherein the scaffold is configured to release the one or more substances in a time-released manner.
   14. The method of any of examples 1 to 13, wherein the scaffold is configured to release the one or more substances based on at least one of movement of a patient in which the scaffold is located or a continuous feedback loop associated with movement of the patient in which the scaffold is located.
   15. The method of any of examples 1 to 14, wherein the scaffold is configured to release an amount of the one or more substances that is proportional to the one or more compressive forces exerted thereon.
   16. The method of any of examples 1 to 15, wherein the scaffold is configured to adjust the release of the one or more substances in response to different compression pressure gradients experienced by the scaffold.
   17. The method of any of examples 1 to 16, wherein the scaffold is configured to be located in the joint space for at least one year.
   18. A meniscal surgical implant, comprising:
a scaffold having a body portion disposed between a first end thereof and a second end thereof, the body portion being sized and shaped for use as one of a replacement or supplement of a meniscus in a patient, the body portion defining an internal cavity that is configured to have one or more substances disposed therein, and the body portion having a porous outer surface configured to allow secretion of one or more substances disposed in the internal cavity to an environment outside of the body portion, the environment being proximate to one of a location
where the meniscus was previously or the meniscus in the patient, wherein the scaffold is configured to release one or more substances from the internal cavity of the body portion in response to one or more compressive forces applied to the body portion by way of anatomy proximate to the one of the location where the meniscus was previously or the meniscus of the patient.
   19. The implant of example 18, wherein the scaffold is C-shaped to mimic the shape of the meniscus.
   20. The implant of example 18 or 19, wherein the scaffold is configured to conform to a space in which it is disposed.
   21. The implant of any of examples 18 to 20, wherein the scaffold is configured to be anchored to the tibia.
   22. The implant of any of examples 18 to 21, wherein at least one of the internal cavity or deformable external cavity is expandable and the body portion further comprises one or more filling openings for receiving at least one of a gas or a liquid to inflate the internal cavity.
   23. The implant of any of examples 18 to 22, further comprising one or more substances disposed within the scaffold, the one or more substances comprising one or more of a medicant or hyaluronic acid (HA).
   24. The implant of example 23, wherein the medicant further comprises at least one of: an analgesic, a non-steroidal inflammatory drug (NSAID), or a non-opioid.
   25. The implant of any of examples 18 to 24, wherein the scaffold is configured to release the one or more substances substantially continuously.
   26. The implant of any of examples 18 to 25, wherein the scaffold is configured to release the one or more substances in a time-released manner.
   27. The implant of any of examples 18 to 26, wherein the scaffold is configured to release the one or more substances based on at least one of movement of a patient in which the scaffold is located or a continuous feedback loop associated with movement of the patient in which the scaffold is located.
   28. The method of any of examples 18 to 27, wherein the scaffold is configured to release an amount of the one or more substances that is proportional to the one or more compressive forces exerted thereon.
   29. The implant of any of examples 18 to 28, wherein the scaffold is configured to adjust the release of the one or more substances in response to different compression pressure gradients experienced by the body portion.
   30. The implant of any of examples 18 to 29, wherein the scaffold further comprises a plurality of chambers formed therein, each of the plurality of chambers being configured to receive at least one substance of the one or more substances.
   31. The implant of example 30, further comprising one or more non-porous walls disposed between chambers of the plurality of chambers such that the at least one substance received in a first chamber of the plurality of chambers is not able to pass through the one or more non-porous walls to a second chamber of the plurality of chambers.
   32. The implant of example 30, wherein the scaffold further comprises a channel that extends from a location proximate to the first end of the body portion towards a location that is more proximate to the second end of the body portion than the first end is, the channel being in fluid communication with one or more branched pathways formed in the body portion, the one or more branched pathways being configured to deliver the one or more substances to the plurality of chambers.
   33. The implant of any of examples 18 to 32, further comprising one or more dispersing openings configured to allow the one or more substances to be released from the internal cavity in response to the one or more compressive forces applied to the body portion by way of anatomy proximate to the one of the location where the meniscus was previously or the meniscus of the patient.
   34. The implant of any of examples 18 to 33, further comprising:
an access port; and
a tube coupled to the access port and providing fluid communication between the access port and the body portion to allow the one or more substances to be introduced into the internal cavity.
   35. The implant of example 34, wherein the access port is configured to be implanted at least one of on or proximate to a skin surface of a patient.
   36. The implant of any of examples 18 to 35, wherein the scaffold is configured to be in fluid communication with a pump to regulate a flow of the one or more substances to the scaffold.
   37. The implant of any of examples 18 to 36, wherein the scaffold is configured to be located at the location of the replacement or supplement of the meniscus in the patient for at least one year.

One skilled in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

1. A surgical method, comprising:
accessing a joint space of a patient that comprises, or previously comprised, a meniscus;
delivering a scaffold to one of replace the meniscus or support the meniscus, the scaffold comprising an internal cavity, and the scaffold having one or more substances disposed in a permeable portion of the internal cavity at least one of during or after it is delivered to the joint space; and
anchoring the scaffold to the tibia,
wherein the scaffold is configured to release the one or more substances from the internal cavity of the scaffold, to the joint space, in response to one or more compressive forces exerted onto the scaffold.

2. The method of claim 1, wherein the internal cavity comprises the permeable portion and a non-permeable portion.

3. The method of claim 2, further comprising:
loading the permeable portion of the internal cavity of the scaffold with the one or more substances.

4. The method of claim 3, wherein loading the permeable portion of the internal cavity of the scaffold with the one or more substances occurs in situ.

5. The method of claim 1, further comprising delivering the one or more substances to the internal cavity after the scaffold is anchored to the tibia.

6. The method of claim 1, further comprising delivering the scaffold into the joint space in an uninflated state.

7. The method of claim 6, further comprising inflating the scaffold to form the implant that accommodates the joint space.

8. The method of claim 1, further comprising coupling an access port to a location on the patient that is remote from the joint space, the access port being in fluid communication with the scaffold to deliver the one or more substances thereto.

9. The method of claim 1, wherein delivering a scaffold further comprises electrospinning the scaffold.

10. (canceled)

11. The method of claim 1, further comprising controlling at least one of a temperature of a material used to form the scaffold, a diameter of a nozzle that delivers a material used to form the scaffold, or a flow rate of a material used to form the scaffold.

12. The method of claim 1, wherein the scaffold is configured to release the one or more substances substantially continuously.

13. The method of claim 1, wherein the scaffold is configured to release the one or more substances in a time-released manner.

14. The method of claim 1, wherein the scaffold is configured to release the one or more substances based on at least one of movement of a patient in which the scaffold is located or a continuous feedback loop associated with movement of the patient in which the scaffold is located.

15. The method of claim 1, wherein the scaffold is configured to release an amount of the one or more substances that is proportional to the one or more compressive forces exerted thereon.

16. The method of claim 1, wherein the scaffold is configured to adjust the release of the one or more substances in response to different compression pressure gradients experienced by the scaffold.

17. The method of claim 1, wherein the scaffold is configured to be located in the joint space for at least one year.

18. A meniscal surgical implant, comprising:
a scaffold having a body portion disposed between a first end thereof and a second end thereof, the body portion being sized and shaped for use as one of a replacement or supplement of a meniscus in a patient, the body portion defining an internal cavity that is configured to have one or more substances disposed therein, and the body portion having a porous outer surface configured to allow secretion of one or more substances disposed in the internal cavity to an environment outside of the body portion, the environment being proximate to one of a location where the meniscus was previously or the meniscus in the patient,
wherein the scaffold is configured to release one or more substances from the internal cavity of the body portion in response to one or more compressive forces applied to the body portion by way of anatomy proximate to the one of the location where the meniscus was previously or the meniscus of the patient.

19. The implant of claim 18, wherein the scaffold is C-shaped to mimic the shape of the meniscus.

20. (canceled)

21. The implant of claim 18, wherein the scaffold is configured to be anchored to the tibia.

22. The implant of claim 18, wherein at least one of the internal cavity or deformable external cavity is expandable and the body portion further comprises one or more filling openings for receiving at least one of a gas or a liquid to inflate the internal cavity.

23. The implant of claim 18, further comprising one or more substances disposed within the scaffold, the one or more substances comprising one or more of a medicant or hyaluronic acid (HA).

24-29. (canceled)

30. The implant of claim 18, wherein the scaffold further comprises a plurality of chambers formed therein, each of the plurality of chambers being configured to receive at least one substance of the one or more substances.

31-37. (canceled)
